(19) United States
(12) Patent Application Publication
(10) Pub. No.: US 2023/0167248 A1
(43) Pub. Date: Jun. 1, 2023

(54) ANTHRACENE-FUNCTIONALIZED DUAL CROSS-LINKED GLYCAN-BASED HYDROGEL WITH REVERSIBLE CROSSLINKING AND A METHOD FOR THEIR PREPARATION

(71) Applicant: KOC UNIVERSITESI, Istanbul (TR)

(72) Inventors: Seda KIZILEL, Istanbul (TR); Afsun SAHIN, Istanbul (TR); Muhammad Anwaar NAZEER, Istanbul (TR); Syeda Rubab BATOOL, Istanbul (TR)

(73) Assignee: KOC UNIVERSITESI, Istanbul (TR)

(21) Appl. No.: 17/916,595

(22) PCT Filed: Apr. 3, 2020
(86) PCT No.: PCT/TR2020/050269

§ 371 Date: Oct. 3, 2022

(51) Int. Cl. C08J 3/075; A61K 47/36

(52) U.S. Cl. CPC C08J 3/075; A61K 47/36; C08J 2305/04

(57) ABSTRACT

An anthracene-functionalized dual cross-linked glycan-based hydrogel with reversible crosslinking, a preparation method thereof, and a use thereof for delivery system of bioactive molecules are provided. The pH responsive and dual crosslinked anthracene incorporated glycan-based hydrogel is prepared via a visible light induced photo-polymerization and a UV light induced photo-dimerization using N-vinyl-2-pyrrolidinone as an accelerator, Eosin-Y as a photo-initiator, and Triethanolamine as a co-initiator.

### CROSS REFERENCE TO THE RELATED APPLICATIONS

This application is the national phase entry of International Application No. PCT/TR2020/050269, filed on Apr. 3, 2020, the entire contents of which are incorporated herein by reference.

### TECHNICAL FIELD

The present invention discloses and claims a novel anthracene-functionalized dual cross-linked glycan-based hydrogel with reversible crosslinking, a preparation method thereof, and a use thereof for delivery system of bioactive molecules.

### BACKGROUND

Glycan-based alginate hydrogels have great potential in biomedicine due to their desirable biological and physical properties. However, low targeting efficiency presents major barrier for clinical translation of these materials.

In one study, ionically cross-linked (CaCl₂) alginate hydrogel was developed and water-induced mechanical transition was studied in these hydrogels (Zhao, Xia, Zhang, Lin, & Wang, 2019). In another study, injectable hydrogels based on aldehyde methacrylate sodium alginate and ethylene-diamine modified gelatin were made via two-step process. In first step, aldehyde groups of alginate were reacted with amino groups of gelatin while in second step, radical reaction was carried out through methacrylate groups of alginate under UV (365 nm) irradiation resulting in increased modulus. (Yuan et al., 2017).

In another recent study authors have developed PEG-anthracene based hydrogel by exploiting dimerization characteristic of anthracene resulting in various modulus (Gunay et al., 2019).

In the United State patent document US20090028946A1, a photo-responsive delivery system by crosslinking polymer backbone with the help of PEG-anthracene based crosslinker was described. The patent shows the capability of dimerization and de-dimerization of anthracene resulting in gel-sol transition.

The prior art on alginate hydrogels involve its electrostatic crosslinking that results in weak mechanical properties or covalently cross-linked hydrogels with no control on light responsive behavior of the hydrogel network. However, the ideal and ‘clinically relevant’ approach would be to design intelligent systems that can respond to alterations in pH and/or light to control the release of bioactive molecules and crosslink density, guide its delivery selectively towards diseased cells/tissues. These needs and other needs are satisfied by the present invention. In this invention, we addressed this urgent need through the design of anthracene-incorporated alginate gel network.

### SUMMARY

According to a first aspect of the invention there is provided a pH responsive anthracene modified glycan-based hydrogel and preparation method thereof.

The other aspect of the present invention is to provide a pH responsive anthracene modified glycan-based hydrogel to obtain tunable crosslink densities.

Another aspect of the present invention is to provide a pH responsive anthracene modified glycan-based hydrogel having unique properties of stiffening and softening behavior that might be useful for cellular behavior at the implanted sight in vivo.

A further aspect of the present invention is to provide a pH responsive anthracene modified glycan-based hydrogel responsive to pH, where it modulates the selective release of the bioactive molecule and water content in hydrogels through alterations in pH.

This present invention provides light-induced control of crosslink density due to anthracene and pH-triggered therapeutics delivery with alginate. The approach would be applicable for systems where multiple controls are required with high precision.

Another aspect of the invention is to provide a pH responsive anthracene modified glycan-based hydrogel, that is prepared through photo-polymerization induced by visible light and photo-dimerization induced by UV light in the presence of an accelerator, a photo-initiator and a co-initiator. In a selected embodiment of the invention the accelerator is selected to be N-vinyl-2-pyrrolidinone (NVP), the photo-initiator is selected to be Eosin-Y and the co-initiator is selected to be Triethanolamine (TEA). The resulting hydrogel turned out to be a functional hydrogel with improved integrity, advanced swelling properties, enhanced swelling properties with less toxicity.

Accordingly, a broad embodiment of the invention is directed to a preparation method which can be used for therapeutic applications.

This object and other objects of this invention become apparent from the detailed discussion of the invention that follows.

### BRIEF DESCRIPTION OF THE DRAWINGS

The present invention is illustrated in the accompanying figures wherein:

FIGS. 1A-1C are an illustration of the key synthesis steps involved in preparation of hydrogel. FIG. 1A Synthesis of 9-Aminoanthracene (9-AA) FIG. 1B Synthesis of MA-alginate FIG. 1C Synthesis of anthracene-MA-alginate.

FIGS. 2A-2C are an illustration of the key steps of hydrogel formation. Photo-polymerization under 514 nm irradiation without FIG. 2A and with FIG. 2B anthracene in the presence of initiator/co-initiator. FIG. 2C Dual cross-linked hydrogel via photo-polymerization of MA and photo-dimerization of anthracene under 365 nm irradiation.

FIGS. 3A-3D are an illustration of FIG. 3A Fourier transform infrared (FTIR) and FIG. 3B UV-Vis spectra of anthracene, 9-NA and 9-AA. Characterization of alginate, MA-alginate and anth-MA-alginate through FIG. 3C FTIR and FIG. 3D UV-Vis.

FIGS. 4A-4D are an illustration of the storage (G′) and loss modulus (G″) as a function of time for. FIG. 4A photo-polymerized MA-alginate and anth-MA-alginate hydrogels (with photo-initiator) and FIG. 4B photo-dimerized anth-MA-alginate (without photo-initiator). FIG. 4C Visual and FIG. 4D schematic demonstration of dimerization/de-dimerization behavior of anth-MA-alginate in the absence of photo-initiator.

FIGS. 5A-5D are an illustration of FIG. 5A optical images and FIG. 5B cyclic pH responsive behavior of hydrogels at acidic and physiological pH. Cumulative drug release % for FIG. 5C MA-alginate and FIG. 5D anth-MA-alginate hydrogels at different pH conditions.

FIGS. 6A-6D are an illustration of surface analysis of FIG. 6A MA-alginate and FIG. 6B anth-MA-alginate while cross-sectional images of FIG. 6C MA-alginate and FIG. 6D anth-MA-alginate.

FIGS. 7A-7F are an illustration of cell viability analysis of hydrogels against FIG. 7A HeLa and FIG. 7B NIH-3T3 cells (****p<0.0001 and **p<0.01). Representative fluorescence images of live/dead cells (FDA, green/PI, red) after 48 h of seeding for NIH-3T3 cells on FIG. 7C MA-alginate, FIG. 7D anth-MA-alginate, and HeLa cells on FIG. 7E MA-alginate and FIG. 7F anth-MA-alginate hydrogel.

### DETAILED DESCRIPTION OF THE EMBODIMENTS

The present invention describes a new stimuli responsive anthracene modified glycan-based hydrogel and preparation method thereof.

This invention is suitable for use in delivery system of bioactive molecule. Stimuli responsive hydrogels have been considered as an agent for delivery of bioactive molecules, especially for bioactive molecules which are needed to be formulated in extended release (eg. sustained, controlled, prolonged) or delayed release pharmaceutical compositions.

Bioactive molecules are molecules that have therapeutic effects on a living organism, tissue or cell. Briefly, bioactive molecule is at least one member of the group consisting of drugs, enzymes, growth factors, hormones, receptors, receptor ligands, adjuvants, genes and antibodies or a mixture of at least two members of these groups.

Unless specified otherwise, the term “delivery system” refers to systems for the delivery of bioactive molecules in vivo and in vitro, and more particularly refers to a delivery system activated by pH or pH alteration.

According to the invention, the hydrogel is a stimuli-responsive hydrogel which is used for extended release bioactive molecule delivery, and preferably the stimulus is pH. pH responsive hydrogels demonstrate reversible swelling and deswelling behavior with a change in pH. This response occurs due to acidic or basic moieties present in hydrogel network which play a role in accepting or releasing protons with alterations in environmental pH.

The term “pH-responsive” refers to the ability of the present invention to alter its configuration when exposed to different pH values. In particular, exposure of the invention to a condition with a specific pH or pH range causes reduction or retention of crosslinking or vice versa.

According to the invention, surprisingly it is found that after anthracene incorporation with photo-polymerized glycan-based hydrogel, improvement in mechanical properties of the hydrogel was observed.

The present invention is a pH responsive and dual crosslinked anthracene incorporated glycan-based hydrogel, which is prepared via visible light induced photo-polymerization and UV light induced photo-dimerization. Preferably, the preparation process is performed in the presence of an accelerator, a photo-initiator and a co-initiator.

The present invention relates to a process for the preparation of pH responsive anthracene modified glycan-based hydrogel. This hydrogel is fabricated from methacrylate-alginate and anthracene using a two-step process. The first step is the synthesis of anthracene-MA-alginate and the second step is the dual crosslinking step.

The first step is an organic synthesis procedure. In the first step, anthrecene-MA-alginate matrice is synthesized according to the below steps:
Synthesis of 9-Aminoanthracene (9-AA)
Synthesis of methacrylated alginate-alginate (MA-alginate)
Synthesis of anthracene-MA-alginate

9-AA is synthesized using two-step procedure as describe previously (Bawa, Alzaraide, & Ben, 2013; Yen & Liou, 2008). MA-alginate is synthesized through esterification reaction as described previously (Chandler et al., 2011). Methacrylated alginate (MA-alginate) through esterification reaction and conjugated it with 9-Aminoanthracene (9-AA) by using EDC/NHS chemistry.

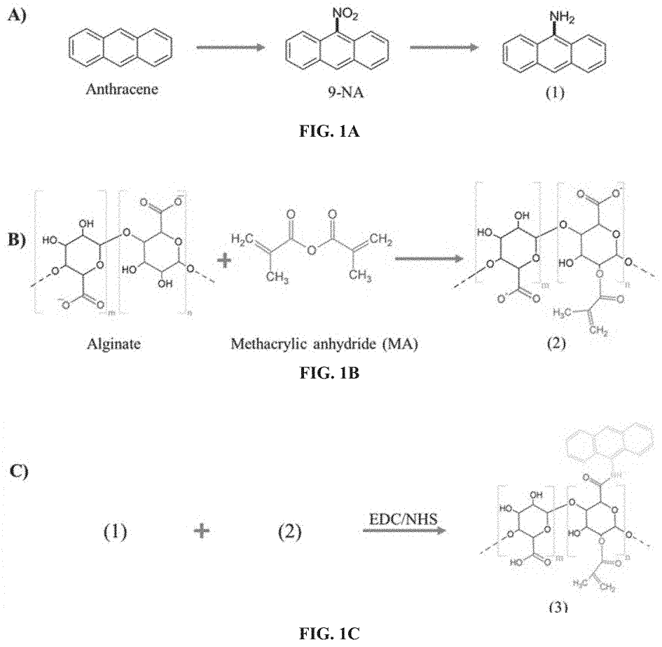

The preparation method of the invention involves, in the first step, the covalent conjugation of anthracene into methacrylate-alginate network (FIGS. 1A-1C). Synthesis of anthracene-MA-alginate is performed by methacrylating alginate (MA-alginate) through esterification reaction and conjugating it with 9-Aminoanthracene (9-AA) by using EDC/NHS chemistry. The pre-polymer solution is obtained in solvent. Preferably, the solvent is Phosphate-buffered saline (PBS).

After providing the pre-polymer solution comprising anthracene-MA-alginate, an accelerator, a photo-initiator and the co-initiator are added to the pre-polymer solution. The second step of the preparation method, dual crosslinking step, is applied on the pre-polymer solution in the presence of an accelerator, a photo-initiator and the co-initiator.

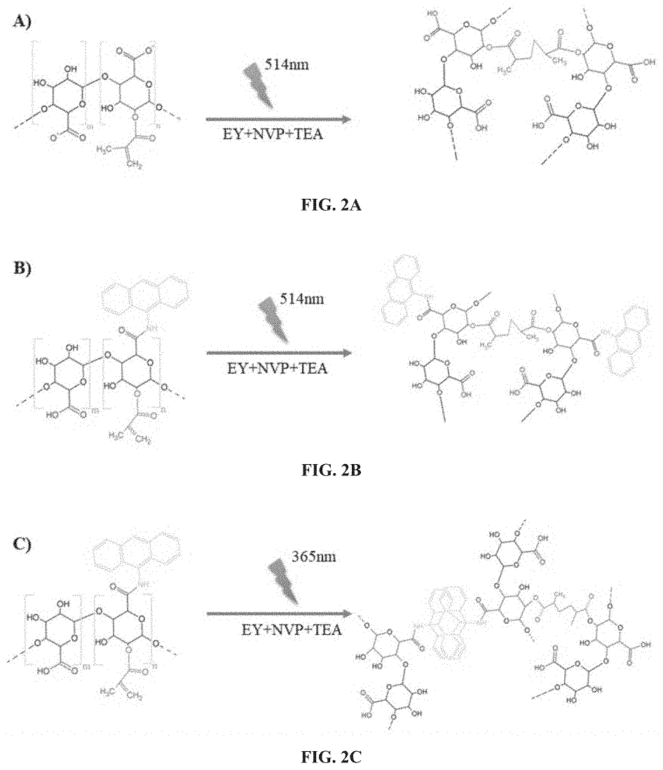

The preparation method of the invention involves, in the second step, dual crosslinking of pre-polymer solution by photo-polymerization of vinyl groups and photo-dimerization of anthracene (FIGS. 2A-2C).

The second step is a dual crosslinking procedure. In the second step, anthracene-functionalized dual cross-linked glycan-based hydrogel with reversible crosslinking is prepared according to the below steps:
free radical photo-polymerization of vinyl groups by visible light and
photo-dimerization of anthracene induced by UV light in the presence of an accelerator, a photo-initiator and a co-initiator.

In a selected embodiment of the invention the accelerator is selected to be N-vinyl-2-pyrrolidinone (NVP), the photo-initiator is selected to be Eosin-Y and the co-initiator is selected to be Triethanolamine (TEA).

Incorporation of anthracene into these gels leads to reversible control on crosslinking and transition between gel/sol states through dimerization/de-dimerization of anthracene groups.

In this invention, crosslink density due to anthracene is controlled by light induced way.

In second step, hydrogel is made by utilizing synthesized materials in the first step. Hydrogel is made through photo-polymerization under 514 nm irradiation with anthracene, where only MA groups of alginate took part in reaction. In parallel, hydrogel is also fabricated by using anthracene and MA functionality of alginate under 365 nm irradiation referred as dual cross-linked hydrogel. This dual cross-linked hydrogel is made through photo-polymerization of MA and photo-dimerization of anthracene. De-dimerization characteristic of anthracene around 254 nm impart unique behavior of reversible control on mechanical properties of hydrogel.

In contrast to reported studies, the present invention shows unique property of stiffening and softening behavior that might be useful for cellular behavior at the implanted sight in vivo. For example, in anthracene based reported studies, hydrogel network breaks and converts to solution upon exposure to 254 nm due to de-dimerization while in the present invention hydrogel will be softened due to the breakage of anthracene crosslinks while maintaining its integrity due to MA crosslinks. This transition from a stiff and highly cross-linked network to more permeable one can be achieved whenever needed in a reversible manner. The same gel can be stiffened again by exposing to 365 nm thus presenting controlled stiffness. In addition, the present invention hydrogels are also responsive to pH, where we are able to modulate the release of the bioactive molecule and water content in hydrogels through alterations in pH. Furthermore, cell viability analysis revealed that growth of cancer cells was comparatively compromised on anthracene conjugated alginate hydrogels with no significant effect on healthy cells.

One object of the present invention is to provide covalently cross-linked alginate which retain hydrogel integrity while at the same time, anthracene moieties play role in softening/stiffening of network through dimerization/de-dimerization.

It is an object of this invention to provide a preparation method of pH responsive and anthracene modified glycan-based hydrogel for selective release of bioactive molecules.

Another object of the invention is the use of the pH responsive and dual crosslinked anthracene incorporated glycan-based hydrogel described before for drug delivery applications such as extended release.

A further object of the invention is a pharmaceutical composition comprising the pH responsive and dual crosslinked anthracene incorporated glycan-based hydrogel described before, an active ingredient and pharmaceutically acceptable excipients.

These examples are intended to representative of specific embodiments of the invention, and are not intended as limiting the scope of the invention.

### Specific Embodiments

In these embodiments, a preparation procedure was applied to provide the anthracene modified glycan-based hydrogel, that specifically pH responsive. After obtaining the candidate hydrogel biomaterial by the preparation method illustrated in FIGS. 1A-1C and FIGS. 2A-2C, experimental studies lead to determine the hydrogel that is specifically pH responsive. Furthermore, characterization of the present invention was studied in vitro.

### EXAMPLES

### Example 1 Covalent Conjugation of Anthracene into Methacrylate-Alginate

Synthesis of 9-Aminoanthracene:
Synthesis of 9-Aminoanthracene (9-NA): anthracene containing methacrylated alginate (anth-MA-alginate) was synthesized via 3 different sub steps. In first, anthracene was converted to 9-Aminoanthracene. (FIG. 1A)
Synthesis of MA-alginate:
Synthesis of MA-alginate: Separately, methacrylic anhydride was reacted with hydroxyl (—OH) groups of alginate to yield MA-alginate. (FIG. 1B)
Synthesis of anthracene-MA-alginate:
Synthesis of anthracene-MA-alginate: In last, amine groups (—NH2) of 9-aminoanthracene were reacted with carboxylate of alginate via carbodiimide chemistry. (FIG. 1C)

### Example 2 Formation of Hydrogel

Hydrogels were made via free radical photo-polymerization under argon laser (argon ion laser, 514 nm, Coherent, Santa Clara, Calif.) using MA-alginate and anth-MA-alginate pre-polymer solutions. Solution was transferred to custom made plastic mold (0.2 cm²) and exposed to laser light for 5 min with a power of 100 mW/cm². The impact of dual crosslinking (photo-polymerization and photo-dimerization) on storage moduli was analyzed by making MA-alginate and anth-MA-alginate hydrogels under UV light (λ=365 nm) equipped with a Rheometer (TA Instruments AR/DHR series, New Castle, Del.) in the presence of photo-initiator (Eosin-Y, (EY)), co-initiator (triethanolamine (TEA)) and accelerator (N-vinyl-2-pyrrolidinone (NVP)). (FIGS. 2A-2C)

### Example 3 Measurements and Comparative Studies

Characterization of Synthesized Materials

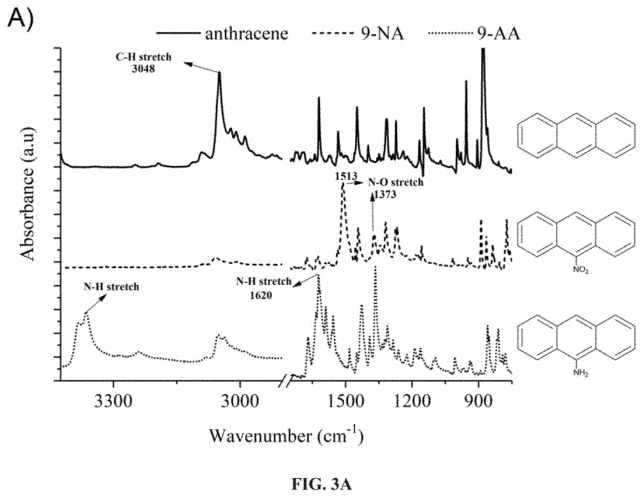

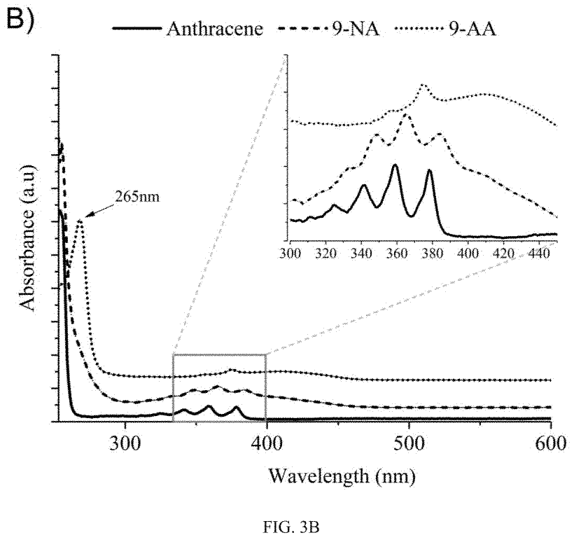

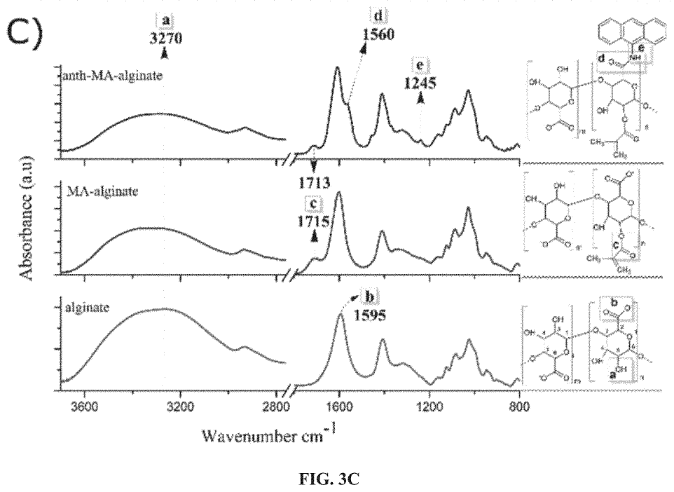

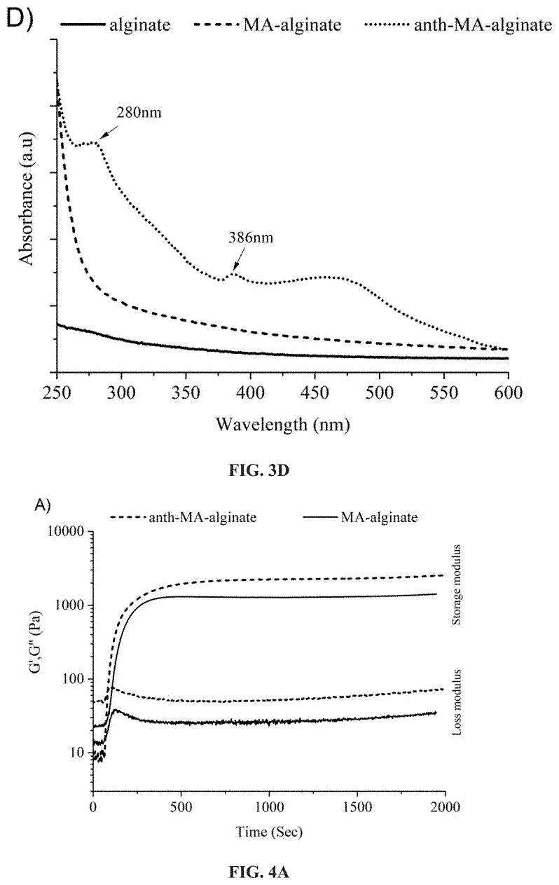

To confirm the successful synthesis of 9-AA, ATR-FTIR, and UV-Vis spectroscopy was performed. In FTIR spectra (FIG. 3A), the sharp band at 3048 cm⁻¹ was assigned to aromatic —CH stretch of anthracene, whereas in 9-NA, there were additional absorption bands at 1513 and 1373 cm⁻¹ positions, which are attributed to the nitro group. In 9-AA, two bands around 3363 and 1620 cm⁻¹ correspond to —NH stretch. The presence of amine groups in spectra confirm the successful synthesis of 9-AA. UV-Vis spectroscopy was carried out to verify the synthesis of 9-AA further. A series of 4 different absorption peaks between 300 to 400 nm (FIG. 3B) correspond to anthracene. In 9-AA spectrum, a shift was observed, and a sharp peak appeared around 265 nm depicts the amine group present in the system. In our study, we synthesized MA-alginate by using excess of MA. MA-alginate has free carboxylate groups, which were utilized to conjugate with amine groups of 9-AA through EDC/NHS chemistry (FIG. 1B). Although anthracene is itself nonpolar, however, when it is introduced in highly hydrophilic polysaccharide (anth-MA-alginate), the overall system remains water soluble. FTIR spectrum of sodium alginate (FIG. 3C) clearly displays the broad band at 3270 cm⁻¹, which corresponds to the —OH stretching band. Another band at 1595 cm⁻¹ position, is related to asymmetric vibration of carbonyl group. Moreover, MA-alginate shows a characteristic band of —C═O of ester group at ˜1715 cm⁻¹ after MA conjugation with alginate. After conjugation of 9-AA with MA-alginate, the band observed at 1560 cm⁻¹ is attributed to —C═O of amide-II, whereas the band at 1245 cm⁻¹ corresponds to —C—N stretch as a result of the reaction of amine groups of anthracene with the carboxylic groups of alginate through carbodiimide chemistry. To investigate the synthesis of anth-MA-alginate further, UV-Vis spectroscopic analysis was carried out (FIG. 3D). Virgin sodium alginate and MA-alginate did not show any absorption peak, however, anth-MA-Alginate displayed absorption peaks at 280 and 386 nm. These peaks are attributed to the three fused benzene rings in anthracene structure and correspond to π_{π}−π_{π} transition.

Visual Demonstration of Dimerization/De-Dimerization Behavior (Rheological Studies)

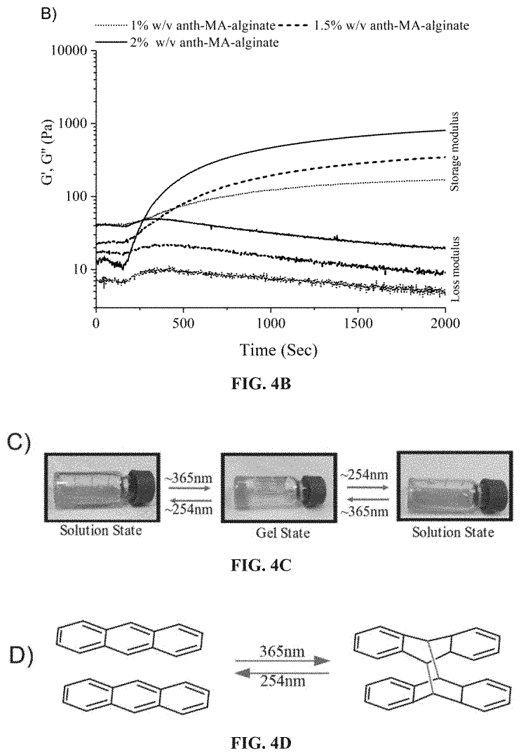

FIGS. 4A-4D are an illustration of the storage (G′) and loss modulus (G″) as a function of time for: photo-polymerized MA-alginate and anth-MA-alginate hydrogels (A) (with photo-initiator) and photo-dimerized anth-MA-alginate (B) (without photo-initiator). Visual demonstration of dimerization/de-dimerization behavior of anth-MA-alginate solution in the absence of photo-initiator (C).

The impact of dual crosslinking (photo-polymerization and photo-dimerization) on storage moduli was analyzed by making MA-alginate and anth-MA-alginate hydrogels under UV light (λ=365 nm) equipped with a Rheometer (TA Instruments AR/DHR series, New Castle, Del.) (FIG. 2C) in the presence of photo-initiator, co-initiator and accelerator. 315 μL of each pre-polymer solution was loaded onto 20 mm quartz plate, working gap was set to be 1000 μm and exposed to UV light with a power of 100 mW/cm². The measurements were carried out in time sweep mode (5 Hz frequency, 1% strain) and storage and loss moduli profiles were recorded as a function of time continuously up to 40 min at ambient temperature. The time required for gelation was estimated through monitoring the elastic and viscous moduli (G′ and G″). For dimerization studies only, pre-polymer solutions of anth-MA-alginate with varying conc. of 1%, 1.5% and 2% w/v in PBS in the absence of photo-initiator and co-initiator were prepared and run.

The storage modulus increased rapidly for both gels due to the formation of effective elastic intermolecular crosslinks (FIG. 4A). It increased sharply within the first 65 seconds, and then continued to increase until 350 seconds. G′ of MA-alginate was leveled off after ˜500 seconds which indicated the completion of photo-crosslinking. However, in case of anth-MA-alginate, photo-crosslinking continued until ˜1000 seconds. Slightly improved modulus observed for anth-MA-alginate can be ascribed to photo-dimerization of anthracene. The contribution of photo-dimerization to higher modulus was further verified through gelation with altered concentration of 1, 1.5 and 2% (w/v) anth-MA-alginate solutions without the use of any photo-initiator, co-initiator and accelerator (FIG. 4B). Increase in modulus can be observed with increasing anthracene concentration, verifying enriched crosslink density due to photo-dimerization of anthracene. Gel made through only photo-dimerization of anthracene was also robust and mechanically stable except for the 1% (w/v) anth-MA-alginate solution. For the gels prepared with 1% and 1.5% (w/v) polymer solutions, distinctive gel point could not be observed due to smaller number of photo-dimerizable moieties present in the system. However, for 2% (w/v) polymer solution, the gel point was clearly observed around 300 seconds. Mechanically robust gels fabricated through photo-dimerization only, can have great significance due to the absence of toxic materials such as photo-initiator and co-initiator etc. As de-dimerization of anthracene happens at ˜254 nm, therefore, cyclic dimerization and de-dimerization can be carried out at 365 and 254 nm depending on the need. This phenomenon was demonstrated repeatedly to show sol-gel transition and vice versa (FIG. 4C). The photo-dimerization of anthracene is based on Diels-Alder cycloaddition reaction in which, [4π+4π] cycloaddition reactions are involved between two anthracene molecules. The [4+4] photocyclodimerization is usually obtained by exposing anthracene containing molecule to UV light (365 nm) and its de-dimerization takes place at 254 nm (FIG. 4D). After dimerization, anthracene is unable to absorb further light at wavelength between 300 and 400 nm, which is a facile way to monitor the completion of dimerization reaction spectrophotometrically.

pH Sensitivity of Hydrogels

To assess the pH sensitivity and mechanical stability, reversible swelling/deswelling behavior of hydrogels was evaluated. Dry hydrogels were pre-weighted, equilibrated in DI water and then incubated in pH 2.0 solution to mimic the stomach environment (artificial gastric fluid). The weight of wet hydrogel was recorded every 30 min for 2 h. Subsequently, hydrogels were re-incubated in pH 7.4 solution to mimic the intestine environment (artificial intestinal fluid) and alteration in weight was recorded after every 30 min for 2 h, and this cycle was repeated three times. The swelling ratio Q, corresponding to the change in weight of hydrogels was calculated by using Eq. 1:

$$Q = \frac{{w}_{s}}{{w}_{d}} \qquad ( 1 )$$

$$Q = \frac{{w}_{s}}{{w}_{d}}$$

where, Wₛ is the weight of swollen or wet hydrogel and W_{d} is the initial weight of dried hydrogel.

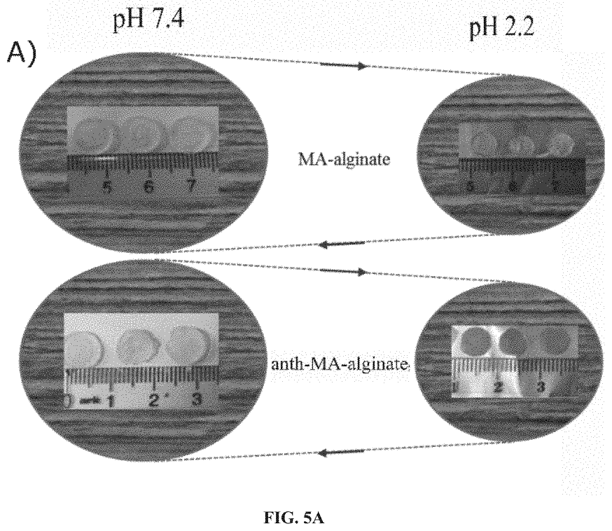

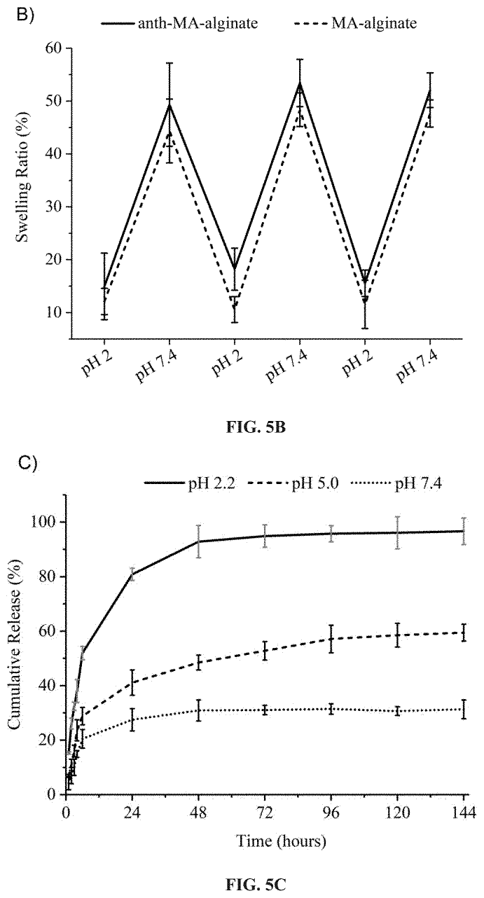

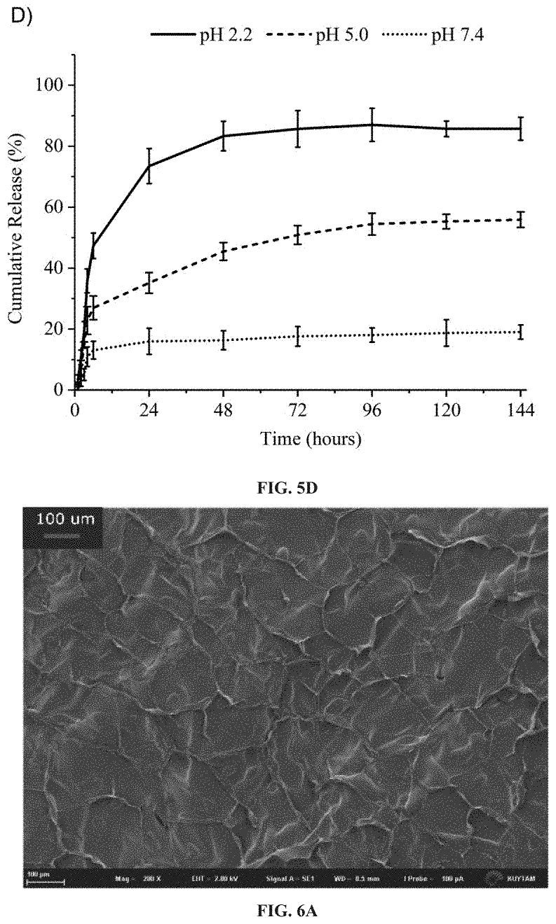

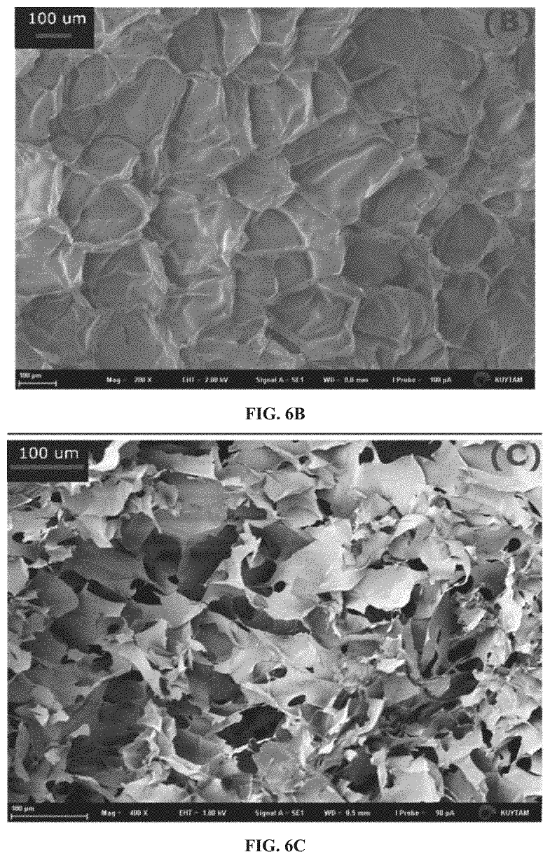

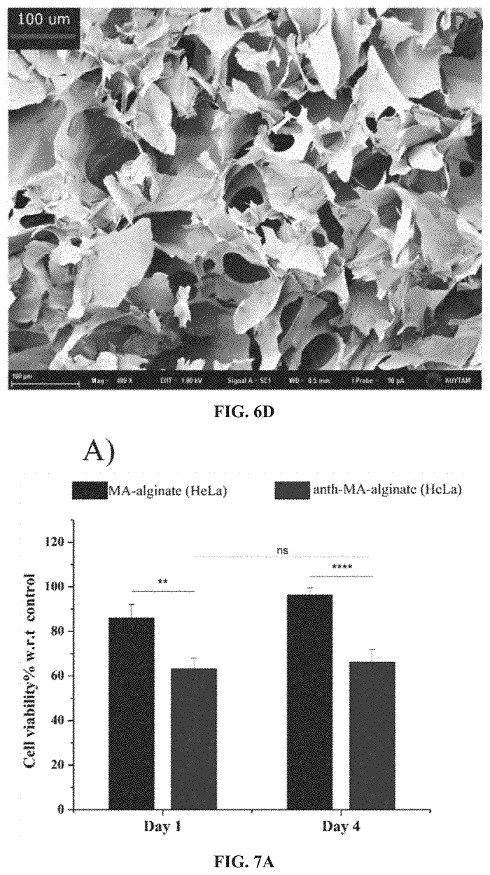

Swelling of alginate hydrogel depends on acidic pendant groups (—COOH) present in the main chain of alginate. At low pH, the carboxylic groups of alginate get protonate causing shrinkage of hydrogel and vice versa at high pH. Hydrogel swelling occurs in steps; first, water diffuses into the hydrogel network instigating loosening up of polymer chains upon hydration and then, relaxation causes expansion in hydrogel. Water within hydrogel network can also interact with the gel matrix either through hydrophilic or hydrophobic interactions. When dry hydrogel absorbs water, hydrophilic interactions take place through hydration of polar groups leading to primary bound water. Upon polar groups' hydration, the gel network swells, and hydrophobic groups also get expose to surrounding water. Subsequently, water interacts with non-polar groups through hydrophobic interactions and is known as hydrophobic or secondary bound water. Moreover, hydrogel can also absorb additional water under the influence of osmotic pressure difference and free water diffuses within hydrogel network. An equilibrium is established between elastic forces and osmotic pressure as explained by Flory and Rehner theory. Both hydrogels exhibited swelling at pH 7.4 and deswelling at acidic pH (FIG. 5B). The swelling ratio of anth-MA-alginate hydrogel was slightly higher than that of MA-alginate hydrogel. This is primarily due to the large mesh size (FIGS. 6A-6D), more water uptake and also possibly due to the presence of hydrophobic moieties (anthracene) which lead to the absorption of additional water through secondary bound water interactions. A visual demonstration was also performed and alteration in hydrogel size was measured after incubation in different pH solutions at specific time points (FIG. 5A). We prepared hydrogels as circular disks (0.9±0.05 cm). MA-alginate hydrogel was clear and transparent, while anth-MA-alginate hydrogel appeared as slightly yellowish and opaque due to the presence of 9-AA within hydrogel structure. In acidic pH (2.2), both hydrogels collapsed, and their size reduced to 0.6±0.1 and 0.8±0.1 cm for MA-alginate and anth-MA-alginate, respectively. However, at physiological pH (7.4), both hydrogels exhibited swelling and the size of both hydrogels was increased to ˜1 cm.

Drug Loading and Release Studies

Doxorubicin (DOX) was used as a model drug for drug loading and release studies. To load drug, individual hydrogel was incubated in 1 mL of drug solution (1 mg/mL) for 24 h at room temperature under constant shaking (75 rpm). Drug loaded hydrogels were washed with DI water 3 times in order to remove free drug molecules present on the surface. The absorbance of the drug solution was measured by nanodrop (Thermoscientific Nanodrop ND100) at 480 nm. DOX solutions with varying concentration were prepared to generate standard calibration curve. Loading efficiency was calculated by using Eq. 2.

Loading Efficiency (%)=(Mₒ−M_{f})/Mₒ×100

Loading Efficiency (%)=(Mₒ−M_{f})/Mₒ×100 (2)

where, Mₒ is the initial mass of DOX in stock solution and M_{f} represents the final mass of DOX remaining in the solution after 24 h.

For drug release studies, three different pH values of 7.4, 5.0 and 2.2 were used in PBS medium. The pH was adjusted by using HCl solution (1N) and DOX loaded hydrogels were incubated at 37±2° C. under constant shaking (100 rpm). Drug release at different time intervals was calculated by using Eq. 3:

Mₜ=Cₜ×V+ECₜ₋₁×VₛMₜ=cₜ×V+ΣCₜ₋₁×Vₛ (3)

where, Mₜ is mass released at time t, Cₜ is DOX concentration at time t, V is the total volume of solution (10 mL), and Vₛ is the sample volume (300 μL). Mₜ value obtained from Eq. 3 can be used to quantify the percent cumulative release of DOX (Eq. 4):

Percent Mass release=(Mₜ/M_{∞})×100

Percent Mass release=(Mₜ/M_{∞})×100

Percent Mass release=(Mₜ−M_{∞})×100 (4)

where, M_{∞} is the total weight of drug loaded into the hydrogel. Sample (300 μL) from each solution was taken for analysis at different time intervals: 1, 2, 3, 4, 6, 24, 48, 72, 96, 120 and 144 h and the final volume of the solution was maintained through addition of the same volume of PBS. The absorbance of the drug was measured at 480 nm by using Nanodrop. Drug concentration was calculated by using standard calibration curve determined through known concentrations of drug solutions.

Drug loading efficiency was found to be 85.3±1.8% and 96.1±0.4% for MA-alginate and anth-MA-alginate hydrogels, respectively. Higher loading efficiency observed for anth-MA-alginate hydrogel is possibly due to the large mesh size and the interaction of DOX with anthracene groups conjugated to hydrogel. The percent DOX release profiles from MA-alginate and anth-MA-alginate hydrogels after 144 h, at physiological pH (7.4) were measured as ˜30% and ˜20%, respectively (FIGS. 5C and 5D). When these hydrogels were incubated at pH 5, percent DOX release from MA-alginate and anth-MA-alginate hydrogels were increased significantly to ˜60% and ˜55%, respectively. Cumulative drug release was also measured in more acidic solution (pH 2.2) in order to reconfirm the pH dependent release profile and the rates were drastically increased to ˜95% and ˜85% for MA-alginate and anth-MA-alginate hydrogels, respectively. These observations verify that the total amount of DOX release is directly influenced by the pH of the medium. For example, after 24 h, the amount of DOX released from MA-alginate hydrogels was ˜80%, ˜40% and ˜25% at pH 2.2, 5.0 and 7.4, respectively (FIG. 5C). On the other hand, anth-MA-alginate hydrogels resulted in comparatively lower amount of release which was around ˜75%, 35% and 16% at pH 2.2, 5.0 and 7.4, respectively (FIG. 5D). Entrapped molecules in a hydrogel network can release through different ways. Diffusion-controlled release mechanism is common in a system that can swell, and the release follows Fick's law of diffusion. Furthermore, the release pattern is also dependent on the solubility of drug in different pH buffers. DOX is highly soluble at lower pH which may augment its fast release from the hydrogel due to the protonation of amine groups present on DOX. However, at pH 7.4, carboxylate of alginate carries a negative charge and amino groups of DOX exhibit a positive charge causing electrostatic interaction between the drug and the network. Hence drug molecules get entrapped and release negligibly at high pH and vice-versa. Furthermore, we have observed that DOX release from anth-MA-alginate hydrogel is slightly lower than the release observed from MA-alginate. This can be attributed to the similarity of DOX structure with anthracene and hence its hydrophobic interaction with the network which in turn hinders its fast release compared to MA-alginate hydrogel. These results show that our system is somehow dependent on pH sensitivity of the hydrogel and anthracene incorporated hydrogel presents more sustained release.

In Vitro Cell Viability and Live/Dead Assay

Cytotoxicity levels of the hydrogel that is obtained via the method of synthesis described in this invention is proved to be comparatively low via cell viability tests.

The survival of NIH-3T3 mouse fibroblast and human cervical cancer cells in the presence of MA-alginate and anth-MA-alginate hydrogels was assessed through cell viability assay. HeLa cells were kindly provided by the school of medicine, Koc University while NIH-3T3 were obtained from ATCC®. Mouse fibroblast and human cervical cancer cells were cultured separately in high-glucose DMEM medium supplemented with 10% FBS, 1% L-glutamine, 1% sodium pyruvate, 1% sodium bicarbonate and 1% penicillin/streptomycin in incubator with 5% CO2 supply at 37° C. Prior to cell seeding, MA-alginate and anth-MA-alginate hydrogels were washed with DI water for 3 days at 37±2° C. and sterilized from both sides through UV exposure for 30 min per each side. Afterwards, HeLa (cell number=20,000) and NIH-3T3 cells (cell number=10,000) were seeded onto hydrogels in 24 well plates by using 800 μL of DMEM medium. Control group was also prepared by seeding the same number of respective cells in well plate in the absence of hydrogels. Cell viability was measured on day-1 and day-4 through adenosine triphosphate (ATP) based CellTiter-Glo (CTG) Assay. ATP standard curve was prepared with known concentrations (1, 0.5, 0.25 and 0.1 μM) in cell culture medium. Cells were incubated in CTG solution while shaking at 150 rpm for 15 min at room temperature. Luminescence from each sample was measured through a plate reader (Biotek, Synergy H1). FDA (5 mg/mL in acetone) and PI (2 mg/mL in PBS) were used to stain live and dead cells respectively. Cells (cell number=50,000) were seeded on each hydrogel in 24 well plate and incubated for 48 h. Subsequently, hydrogels were placed in a petri dish and suspended in 2 mL of PBS. The cells were stained with FDA/PT solutions (60 μL of PT and 10 μL of FDA) in dark for 5 min at room temperature. Hydrogels were rinsed with PBS and examined under fluorescent microscope (Nikon eclipse Ni).

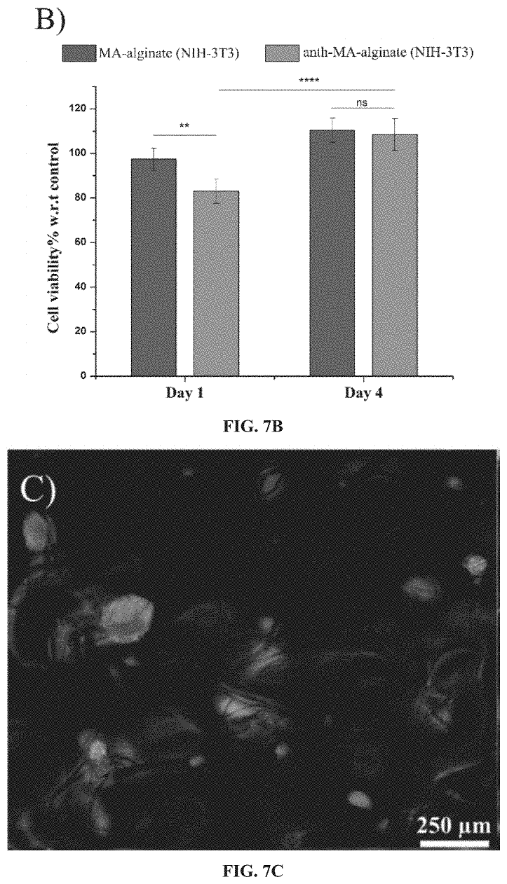

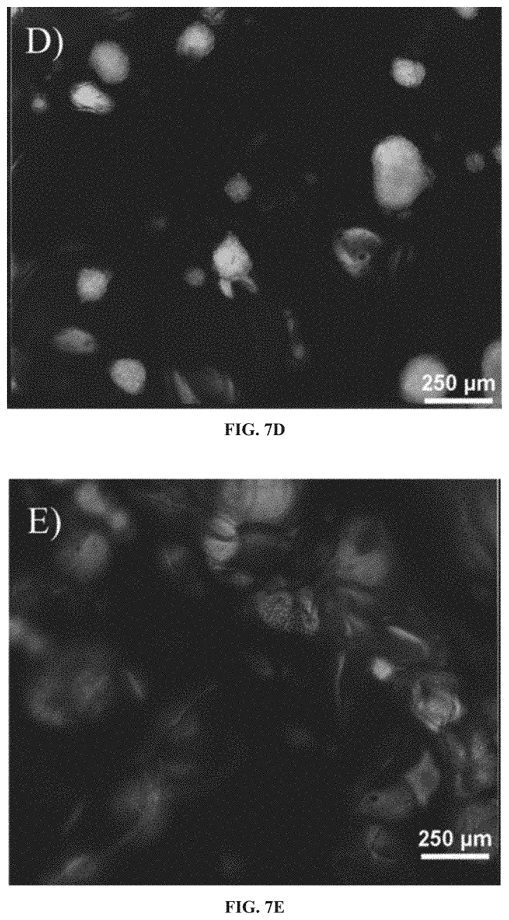

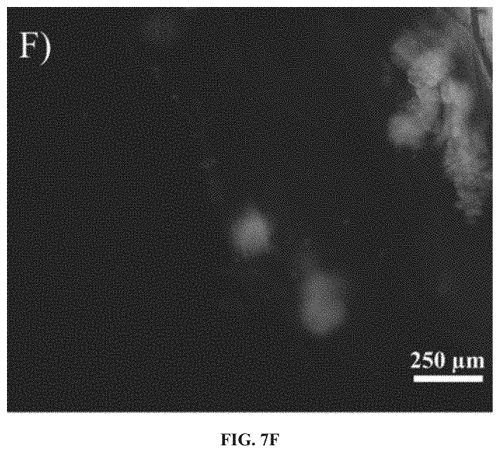

Cytocompatibility of MA-alginate and anth-MA-alginate hydrogels was investigated through cell viability assay (FIGS. 6A-6D). Percent survival of HeLa cells in MA-alginate hydrogel was measured as ˜86% on day-1 and ˜96% on day-4. Slightly lower viability on day-1 might be due to the anticancer property of alginate. On the other hand, for anth-MA-alginate hydrogel, viability of HeLa cells significantly reduced to ˜65% (**p<0.01 for day-1 and ****p<0.0001 for day-4) (FIG. 7A). The compromised HeLa cell proliferation and hence lower viability is probably due to the presence of anthracene derivative within our hydrogel structure. To check the impact of anthracene on healthy cells, we also investigated the viability of NIH-3T3 fibroblast cells. Percent cell survival observed for NIH-3T3 fibroblast cells in MA-alginate and anth-MA-alginate hydrogels was >95% and ˜85% on day-1, respectively (FIG. 7B). Similarly, both kind of hydrogels did not compromise the proliferation rate of fibroblast cells on day 4, where viability measured was >100% compared to the control group. These observations suggest that presence of anthracene derivative compromised the growth of cancer cells and endorse our hypothesis. In our previous study, PEG hydrogel coated nanocarriers were loaded with DOX, where the effect of DOX release from enzymatically degradable network influenced fibroblast and HeLa cell viability (Nazli, Demirer, Yar, Acar, & Kizilel, 2014). HeLa cells were strongly affected than healthy fibroblast cells, probably due to the fewer number of integrins in case of fibroblast cells. Cell seeded hydrogels were further visualized through Live/Dead assay (FDA/PI staining) under fluorescence microscope (FIGS. 7C-7F). Predetermined number of fibroblast and HeLa cells suspended in medium were seeded on hydrogels. The green color represents live cells (FDA stained) while dead cells express red color (PI staining). Fibroblast cells showed good viability on both types of hydrogels and most of the cells were alive even after 48 h (FIGS. 7C and 7D). On the other hand, HeLa cells exhibited a compromised growth on MA-alginate in general and particularly on anth-MA-alginate hydrogel (FIGS. 7E and 7F) based dead cells observed. These results are also consistent with our cell viability assay. Cells demonstrated spherical morphology due to the lack of integrin engagement sites available in alginate to sense mechanical stiffness within the microenvironment. Furthermore, NIH-3T3 cells are very prone to form spherical morphology if they do not get a suitable place for adhesion and due to the spongy nature of hydrogel, can form aggregates as well. Cell-aggregation in HeLa cells is also a reported phenomenon but mechanism is unknown. Besides many possible explanations regarding aggregate formation, porosity of hydrogel might be a significant reason. Pores in a 3D hydrogel may allow cell migration and so interaction with neighboring cells happens depending on their physical locations and the secretory molecules surrounding them. Further, crosstalk permitting enhances ECM protein synthesis, which in turn can promote cell aggregation. Consequently, few cells remain on gel surface and rest of the cells migrate towards the interior domains within the hydrogel through the pores and finally form spheres.

### REFERENCES

Bawa, R. A., Alzaraide, E. M., & Ben, N. N. (2013). Synthesis and electric conductivity study of the new azo dye 4-(9-anthrylazo) phenol. Int. Journal of Engineering Research and Application, 3, 1-5.
Chandler, E. M., Berglund, C. M., Lee, J. S., Polacheck, W. J., Gleghorn, J. P., Kirby, B. J., & Fischbach, C. (2011). Stiffness of photocrosslinked RGD-alginate gels regulates adipose progenitor cell behavior. Biotechnology and bioengineering, 108(7), 1683-1692.
Günay, K. A., Ceccato, T. L., Silver, J. S., Bannister, K. L., Bednarski, O. J., Leinwand, L. A., & Anseth, K. S. (2019). PEG-Anthracene Hydrogels as an On-Demand Stiffening Matrix To Study Mechanobiology. Angewandte Chemie, 131(29), 10017-10021.
Nazli, C., Demirer, G. S., Yar, Y., Acar, H. Y., & Kizilel, S. (2014). Targeted delivery of doxorubicin into tumor cells via MMP-sensitive PEG hydrogel-coated magnetic iron oxide nanoparticles (MIONPs). Colloids and Surfaces B: Biointerfaces, 122, 674-683.
Yen, H. j., & Liou, G. S. (2008). Novel thermally stable triarylamine-containing aromatic polyamides bearing anthrylamine chromophores for highly efficient green-light-emitting materials. Journal of Polymer Science Part A: Polymer Chemistry, 46(22), 7354-7368.
Yuan, L., Wu, Y., Gu, Q.-s., El-Hamshary, H., El-Newehy, M., & Mo, X. (2017). Injectable photo crosslinked enhanced double-network hydrogels from modified sodium alginate and gelatin. International journal of biological macromolecules, 96, 569-577.
Zhao, X., Xia, Y., Zhang, X., Lin, X., & Wang, L. (2019). Design of mechanically strong and tough alginate hydrogels based on a soft-brittle transition. International journal of biological macromolecules, 139, 850-857.

What is claimed is:

1. A pH responsive and dual crosslinked anthracene incorporated glycan-based hydrogel, prepared via a visible light induced photo-polymerization and a UV light induced photo-dimerization.

2. The pH responsive and dual crosslinked anthracene incorporated glycan-based hydrogel according to claim 1, wherein an accelerator, a photo-initiator, and a co-initiator are used in a preparation.

3. The pH responsive and dual crosslinked anthracene incorporated glycan-based hydrogel according to claim 2, wherein the accelerator is N-vinyl-2-pyrrolidinone, the photo-initiator is Eosin-Y, and the co-initiator is Triethanolamine.

4. The pH responsive and dual crosslinked anthracene incorporated glycan-based hydrogel according to claim 1, wherein a method of a preparation comprises a process comprising the following steps:
(a) providing a pre-polymer solution comprising anthracene-MA-alginate,
(b) adding an accelerator N-vinyl-2-pyrrolidinone, a photo-initiator Eosin-Y, and a co-initiator Triethanolamine to the pre-polymer solution,
(c) conducting a dual crosslinking of the pre-polymer solution via the visible light induced photo-polymerization of MA-alginate and the UV light induced photo-dimerization of anthracene.

5. A method of preparing a pH responsive and dual crosslinked anthracene incorporated glycan-based hydrogel via a visible light induced photo-polymerization and a UV light induced photo-dimerization.

6. The method of preparing the pH responsive and dual crosslinked anthracene incorporated glycan-based hydrogel according to claim 5, wherein N-vinyl-2-pyrrolidinone is used as an accelerator, Eosin-Y is used as a photo-initiator, and Triethanolamine is used as a co-initiator.

7. The method of preparing the pH responsive and dual crosslinked anthracene incorporated glycan-based hydrogel according to claim 5, wherein the method is comprises a process comprising the following steps:
(a) providing a pre-polymer solution comprising anthracene-MA-alginate,
(b) adding an accelerator N-vinyl-2-pyrrolidinone, a photo-initiator Eosin-Y, and a co-initiator Triethanolamine to the pre-polymer solution,
(c) conducting a dual crosslinking of the pre-polymer solution via the visible light induced photo-polymerization of MA-alginate and the UV light induced photo-dimerization of anthracene.

8. The pH responsive and dual crosslinked anthracene incorporated glycan-based hydrogel according to claim 1, wherein the pH responsive and dual crosslinked anthracene incorporated glycan-based hydrogel is used as a drug delivery agent for an extended release.

9. A pharmaceutical composition comprising the pH responsive and dual crosslinked anthracene incorporated glycan-based hydrogel according to claim 1, an active ingredient, and pharmaceutically acceptable excipients.

10. The pH responsive and dual crosslinked anthracene incorporated glycan-based hydrogel according to claim 2, wherein a method of a preparation comprises a process comprising the following steps:
(a) providing a pre-polymer solution comprising anthracene-MA-alginate,
(b) adding the accelerator N-vinyl-2-pyrrolidinone, the photo-initiator Eosin-Y, and the co-initiator Triethanolamine to the pre-polymer solution,
(c) conducting a dual crosslinking of the pre-polymer solution via the visible light induced photo-polymerization of MA-alginate and the UV light induced photo-dimerization of anthracene.

11. The pH responsive and dual crosslinked anthracene incorporated glycan-based hydrogel according to claim 3, wherein a method of a preparation comprises a process comprising the following steps:
(a) providing a pre-polymer solution comprising anthracene-MA-alginate,
(b) adding the accelerator N-vinyl-2-pyrrolidinone, the photo-initiator Eosin-Y, and the co-initiator Triethanolamine to the pre-polymer solution,
(c) conducting a dual crosslinking of the pre-polymer solution via the visible light induced photo-polymerization of MA-alginate and the UV light induced photo-dimerization of anthracene.

12. The method of preparing the pH responsive and dual crosslinked anthracene incorporated glycan-based hydrogel according to claim 6, wherein the method comprises a process comprising the following steps:
(a) providing a pre-polymer solution comprising anthracene-MA-alginate,
(b) adding the accelerator N-vinyl-2-pyrrolidinone, the photo-initiator Eosin-Y, and the co-initiator Triethanolamine to the pre-polymer solution,
(c) conducting a dual crosslinking of the pre-polymer solution via the visible light induced photo-polymerization of MA-alginate and the UV light induced photo-dimerization of anthracene.

13. The pH responsive and dual crosslinked anthracene incorporated glycan-based hydrogel according to claim 2, wherein the pH responsive and dual crosslinked anthracene incorporated glycan-based hydrogel is used as a drug delivery agent for an extended release.

14. The pH responsive and dual crosslinked anthracene incorporated glycan-based hydrogel according to claim 3, wherein the pH responsive and dual crosslinked anthracene incorporated glycan-based hydrogel is used as a drug delivery agent for an extended release.

15. The pH responsive and dual crosslinked anthracene incorporated glycan-based hydrogel according to claim 4, wherein the pH responsive and dual crosslinked anthracene incorporated glycan-based hydrogel is used as a drug delivery agent for an extended release.

16. The pharmaceutical composition according to claim 9, wherein an accelerator, a photo-initiator, and a co-initiator are used in a preparation of the pH responsive and dual crosslinked anthracene incorporated glycan-based hydrogel.

17. The pharmaceutical composition according to claim 16, wherein the accelerator is N-vinyl-2-pyrrolidinone, the photo-initiator is Eosin-Y, and the co-initiator is Triethanolamine.

18. The pharmaceutical composition according to claim 9, wherein a method of a preparation of the pH responsive and dual crosslinked anthracene incorporated glycan-based hydrogel comprises a process comprising the following steps:
(a) providing a pre-polymer solution comprising anthracene-MA-alginate,
(b) adding the accelerator N-vinyl-2-pyrrolidinone, the photo-initiator Eosin-Y, and the co-initiator Triethanolamine to the pre-polymer solution,
(c) conducting a dual crosslinking of the pre-polymer solution via the visible light induced photo-polymerization of MA-alginate and the UV light induced photo-dimerization of anthracene.
